(19) United States
(12) Patent Application Publication
(10) Pub. No.: US 2025/0134118 A1
(43) Pub. Date: May 1, 2025

(54) FRESHNESS MAINTENANCE SYSTEM

(71) Applicant: DENSO CORPORATION, Kariya-city (JP)

(72) Inventors: Naohisa NIIMI, Kariya-city (JP); Minekazu SAKAI, Kariya-city (JP); Norihiro KURUMADO, Kariya-city (JP); Tomohide ARIKI, Kariya-city (JP)

(21) Appl. No.: 19/010,738

(22) Filed: Jan. 6, 2025

Related U.S. Application Data
(63) Continuation of application No. PCT/JP2023/024587, filed on Jul. 3, 2023.

(30) Foreign Application Priority Data
Jul. 11, 2022 (JP) 2022-111344

(51) Int. Cl. A23B 2/53; A23B 2/00; A23B 2/704

(52) U.S. Cl. CPC A23B 2/53; A23B 2/003; A23B 2/704

(57) ABSTRACT

A freshness maintenance system includes a freshness detection unit that detects the freshness of an agricultural product by detecting ethylene gas, and a maturation suppression unit that irradiates light having a sterilizing effect or generates an electromagnetic wave. A processing unit of the freshness maintenance system controls the maturation suppression unit based on the freshness detected by the freshness detection unit to suppress the freshness of the agricultural product.

### CROSS REFERENCE TO RELATED APPLICATION

The present application is a continuation application of International Patent Application No. PCT/JP2023/024587 filed on Jul. 3, 2023, which designated the U.S. and claims the benefit of priority from Japanese Patent Application No. 2022-111344 filed on Jul. 11, 2022. The entire disclosures of all of the above applications are incorporated herein by reference.

### TECHNICAL FIELD

The disclosure in this specification relates to a freshness maintenance system that maintains the freshness of agricultural product.

### BACKGROUND ART

A conceivable technique teaches a freshness maintenance system of agricultural product for maintaining the freshness of agricultural product.

### SUMMARY OF INVENTION

According to an example, a freshness maintenance system may include a freshness detection unit that detects the freshness of an agricultural product by detecting ethylene gas, and a maturation suppression unit that irradiates light having a sterilizing effect or generates an electromagnetic wave. A processing unit of the freshness maintenance system controls the maturation suppression unit based on the freshness detected by the freshness detection unit to suppress the freshness of the agricultural product.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a control configuration diagram of a freshness maintenance system according to a first embodiment;

FIG. 2 is a diagram showing the configuration of a freshness maintenance system according to a first example;

FIG. 3 is a diagram showing the configuration of a freshness maintenance system according to a second example;

FIG. 4 is a diagram showing the configuration of a freshness maintenance system according to a third example;

FIG. 5 is a diagram showing the configuration of a freshness maintenance system according to a fourth example;

FIG. 6 is a diagram showing the configuration of a freshness maintenance system according to a fifth example;

FIG. 7 is a diagram showing the configuration of a freshness maintenance system according to a sixth example;

FIG. 8 is a diagram showing the freshness maintenance system accommodated in a box;

FIG. 9 is a diagram showing an agricultural product and a freshness maintenance system;

FIG. 10 is a flow chart showing the operation of the freshness maintenance system;

FIG. 11 is a control configuration diagram of a freshness maintenance system according to a second embodiment;

FIG. 12 is a flow chart showing the operation of the freshness maintenance system;

FIG. 13 is a control configuration diagram of a freshness maintenance system according to a third embodiment;

FIG. 14 is a diagram showing a freshness maintenance system and an agricultural product according to a third embodiment;

FIG. 15 is a flowchart showing the control of step S120;

FIG. 16 is a control configuration diagram of a freshness maintenance system according to a fourth embodiment;

FIG. 17 is a flow chart showing the operation of the freshness maintenance system; and

FIG. 18 is a flowchart showing the operation of the freshness maintenance system of the fifth embodiment.

### DETAILED DESCRIPTION

A conceivable technique describes a technique for maintaining the freshness by irradiating agricultural products on a conveyor belt with light in the near infrared range and light in the ultraviolet range. The conceivable technique has room for improvement in terms of maintaining the freshness during transportation until the agricultural product reaches the customer.

The purpose of the embodiments is to provide a freshness maintenance system that can maintain the freshness of agricultural produce.

A freshness maintenance system according to the present embodiments includes: a freshness detection unit that detects a specific gas to detect a freshness of an agricultural product; a maturation suppression unit that irradiates light having a sterilizing effect; and a control unit that controls the maturation suppression unit based on the freshness detected by the freshness detection unit to suppress a progress of maturing of the agricultural product.

A freshness maintenance system according to the present embodiments includes: a freshness detection unit that detects a specific gas to detect a freshness of an agricultural product; a maturation suppression unit that generates an electromagnetic wave; and a control unit that controls the maturation suppression unit based on the freshness detected by the freshness detection unit to suppress a progress of maturing of the agricultural product.

These freshness maintenance system can detect the maturation degree and spoiled smell of the agricultural product using the freshness detection unit, and can irradiate the light to suppress the progress of maturing according to the detected freshness state. Thus, it is possible to suppress the progress of maturing during storage of the agricultural product or display in a store. Therefore, this technique can provide a freshness maintenance system that can maintain the freshness of the agricultural product.

The following describes embodiments for carrying out the present disclosure with reference to the drawings. In each embodiment, parts corresponding to the elements described in the preceding embodiments are denoted by the same reference numerals, and redundant explanation may be omitted. When only a part of the configuration is described in each embodiment, another embodiment described previously may be applied to the other parts of the configuration. It may be possible not only to combine parts the combination of which is explicitly described in an embodiment, but also to combine parts of respective embodiments the combination of which is not explicitly described if any obstacle does not especially occur in combining the parts of the respective embodiments.

### First Embodiment

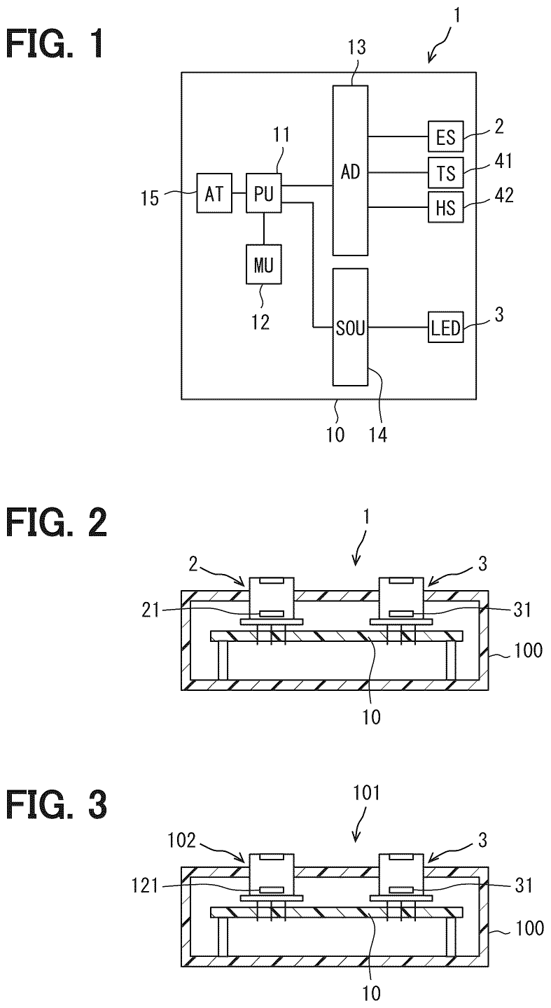

A first embodiment teaching an example of a freshness maintenance system will be described with reference to FIGS. 1 to 10. The freshness maintenance system monitors the freshness of an agricultural product and implements control to suppress the progress of maturing in order to maintain the freshness. The freshness maintenance system can be applied to the management of agricultural product from shipment to transportation, the management in storage environment such as a warehouse, and in store sales area for agricultural product. The freshness maintenance system is particularly useful for room temperature management. The freshness maintenance system can also be applied to the management of agricultural product in an environment where the agricultural product may mature or spoil. The freshness maintenance system can be applied to agricultural product stored in a cardboard box, a container, and the like. The freshness maintenance system can also be applied to agricultural product that is not accommodated in a container but is placed in an open space.

As shown in FIG. 1, the freshness maintenance system 1 includes a freshness detection unit 2 capable of detecting the freshness of agricultural product, and a maturation suppression unit 3 capable of suppressing the degree of maturation of the agricultural product. The freshness detection unit 2 detects a specific gas to detect a freshness of an agricultural product. The maturation suppression unit 3 is a generation source that emits light having a sterilizing effect. Furthermore, the freshness maintenance system 1 may be configured to include a temperature sensor 41 capable of detecting the temperature of the atmosphere or the temperature of the agricultural product, and a humidity sensor 42 capable of detecting the relative humidity of the atmosphere.

The freshness maintenance system 1 includes an acquisition unit 13, an irradiation unit 14, a memory unit 12, a processing unit 11 and an output unit 15. The freshness maintenance system 1 includes a circuit board 10 that constitutes a control circuit and includes a CPU. On the circuit board 10, one or more electronic components having the functions of an acquisition unit 13, an irradiation unit 14, a memory unit 12, a processing unit 11, and an output unit 15 are mounted. The circuit board 10 is supplied with electric power from a battery or an external power source. This power supply drives the various electronic components. The processing unit 11 functions as a control unit of the freshness maintenance system 1 that controls the maturation suppression unit 3 based on the detected freshness of the agricultural product.

In the drawing, the acquisition unit 13 is represented as AD, the irradiation unit 14 is represented as SOU, the memory unit 12 is represented as MU, the processing unit 11 is represented as PU, and the output unit 15 is represented as AT. The detection value detected by the freshness detection unit 2 is input to the acquisition unit 13. When the temperature sensor 41 and the humidity sensor 42 are provided, the acquisition unit 13 receives temperature information detected by the temperature sensor 41 and humidity information detected by the humidity sensor 42. In the drawing, the freshness detection unit 2 is represented as ES, the temperature sensor 41 is represented as TS, the humidity sensor 42 is represented as HS, and the maturation suppression unit 3 is represented as LED.

The irradiation unit 14 is electrically connected to the maturation suppression unit 3. The maturation suppression unit 3 irradiates the agricultural product with irradiation light capable of suppressing the degree of maturation of the agricultural product. The maturation suppression unit 3 is an ultraviolet ray irradiation functional unit capable of irradiating, for example, UV-A or UV-B. The UV-A is a long-wavelength ultraviolet ray with a wavelength of 315 nm to 400 nm. The UV-B is ultraviolet light with a medium wavelength of 280 nm to 315 nm. A control signal for controlling the wavelength and the irradiation amount of the ultraviolet light is output from the irradiation unit 14 to the maturation suppression unit 3.

The maturation suppression unit 3 may be configured to have an irradiation unit capable of irradiating infrared light or near-infrared light, and can provide a sterilizing effect on the agricultural product. Moreover, the maturation suppression unit 3 may be configured to provide ozone, to irradiate light onto a photocatalyst, or to provide an electromagnetic wave. The ozone sterilizes the agricultural product by destroying a cell membrane of bacteria with the strong oxidizing power of the ozone. For example, the ozone is removed by converting ethylene to ethylene oxide, and the like.

Sterilization using a photocatalyst is performed by, for example, applying light to the photocatalyst to break down an impurity such as organic matter and remove bacteria and other germs. For example, when the ultraviolet light is irradiated onto the photocatalyst from the ultraviolet light irradiation unit and ethylene gas flows along the photocatalyst, the ultraviolet light causes photocatalytic activity in the metal oxide coated on the photocatalyst, and the ethylene gas is decomposed and removed.

The memory unit 12 is a non-transitory tangible storage medium that non-transitory stores programs and data that can be read by a computer or a processor. The memory unit 12 includes a volatile memory and a nonvolatile memory. The memory unit 12 stores a program for the processing unit 334 to execute a calculation process. The memory unit 12 temporarily stores data when the processing unit 11 executes the calculation process. The memory unit 12 stores various data to be input to the acquisition unit 13 and the acquisition time of the various data.

The output unit 15 wirelessly transmits the processing result by the processing unit 11 to an external device or the cloud system. The external device is configured to display the information acquired from the output unit 15 on a display screen or to notify the information by sound or the like. A user or an administrator can acquire information from the freshness maintenance system 1 as an alert via an external device or the cloud system. The external device is, for example, a mobile terminal such as a smartphone, or a personal computer. Furthermore, the freshness maintenance system 1 may be configured to display the results of processing by the processing unit 11 on a display screen of a device constituting the system or to notify the user by sound or the like.

The freshness maintenance system can be manufactured in a variety of configurations. The freshness maintenance system includes a casing 100. The freshness maintenance system includes a freshness detection unit 2 and a maturation suppression unit 3, which are exposed to the outside from the casing 100. Hereinafter, first to sixth examples of the configuration of the freshness maintenance system will be described with reference to FIGS. 2 to 8.

FIG. 2 shows the configuration of a freshness maintenance system 1 according to a first example. The casing 100 shown in FIG. 2 accommodates a circuit board 10 to which a semiconductor chip 21 functioning as the freshness detection unit 2 and a semiconductor chip 31 functioning as the maturation suppression unit 3 are connected. The freshness detection unit 2 is a variety of sensors capable of detecting the freshness of the agricultural product. The freshness detection unit 2 has a built-in semiconductor chip 21 and is packaged in the casing 100. The maturation suppression unit 3 has a built-in semiconductor chip 31 and is packaged in the casing 100. The semiconductor chip 21 is a sensor element that functions as a smell sensor or an ethylene sensor. The semiconductor chip 31 functions as an ultraviolet light irradiation unit capable of irradiating the UV-A light and the UV-B light. The freshness maintenance system 1 has a configuration in which a single semiconductor chip functioning as a single sensor is accommodated in a single casing. Moreover, the irradiating portion of the maturation suppression unit 3 may be configured to include a lens in order to improve the irradiation intensity of light or adjust the irradiation range.

The ethylene sensor is one of the freshness detection units that detects a specific gas, and detects the ethylene concentration inside a box or storage room, or the ethylene concentration in the atmosphere as the measurement target. The ethylene sensor is, for example, a semiconductor-based gas sensing device. Ethylene generated from the agricultural product may have a negative impact on the freshness. Ethylene accelerates the maturation of the product and may cause the product to over-mature the product if the storage period of the product is long. When the freshness maintenance system is equipped with an ethylene sensor, the freshness maintenance system can detect the freshness of the product by measuring the degree of spoil or the degree of maturation of the produce by detecting the ethylene concentration.

The smell sensor is one type of the freshness detection units that detects a specific gas, and detects a specific smell component using, for example, a metal oxide semiconductor. When the freshness maintenance system is equipped with a smell sensor, the freshness maintenance system can detect the freshness of the product by measuring the strength of a smell component generated from the product by detecting a specific smell component.

FIG. 3 shows the configuration of a freshness maintenance system 101 according to a second example. The casing 100 shown in FIG. 3 accommodates a circuit board 10 to which a semiconductor chip 121 functioning as the freshness detection unit 102 and a semiconductor chip 31 functioning as the maturation suppression unit 3 are connected. The freshness detection unit 102 has a built-in semiconductor chip 121 and is packaged in the casing 100. The semiconductor chip 121 is a sensor element that functions as a smell sensor and an ethylene sensor. The semiconductor chip 31 functions as an ultraviolet light irradiation unit capable of irradiating the UV-A light and the UV-B light. The freshness maintenance system 101 has a configuration in which a single semiconductor chip functioning as two sensors is accommodated in a single casing.

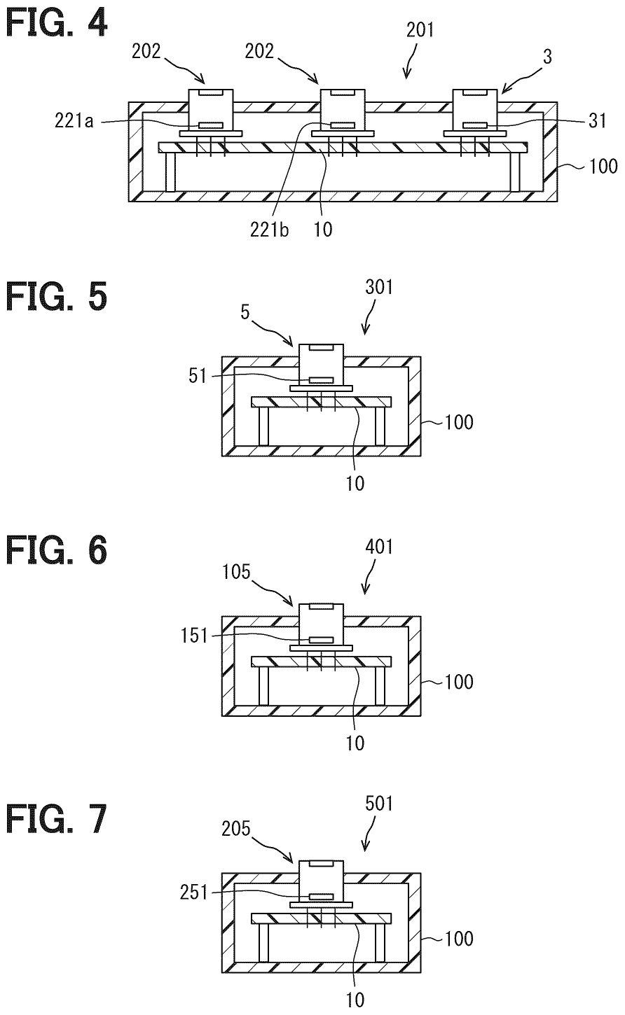

FIG. 4 shows the configuration of a freshness maintenance system 201 according to a third example. The housing 100 shown in FIG. 4 accommodates a semiconductor chip 221a and a semiconductor ship 221b functioning as the freshness detection unit 202 and a circuit board 10 to which the semiconductor chip 31 functioning as the maturation suppression unit 3 are connected. The semiconductor chip 221a is a sensor element that functions as a smell sensor. The semiconductor chip 221b is a sensor element that functions as an ethylene sensor. The freshness detection unit 202 has a built-in semiconductor chip 221a and is packaged in the casing 100. The other freshness detection unit 202 has a built-in semiconductor chip 221b and is packaged in the casing 100. The freshness maintenance system 201 includes two casings, and each casing accommodates one semiconductor chip that functions as one sensor.

FIG. 5 shows the configuration of a freshness maintenance system 301 according to a fourth example. The housing 100 shown in FIG. 5 accommodates a circuit board 10 which is connected to a semiconductor chip 51 that constitutes the freshness maintenance unit 5 that functions as a freshness detection unit and a maturation suppression unit. The semiconductor chip 51 is a sensor element that functions as a smell sensor, an ethylene sensor, and an ultraviolet light irradiation source. In other words, the ethylene sensor, the smell sensor, and the ultraviolet light irradiation unit are mounted on a single semiconductor chip. The freshness maintenance unit 5 includes a built-in semiconductor chip 51 having three functions and is packaged in the casing 100. The freshness maintenance system 301 is configured to include one casing that accommodates a one-chip semiconductor that functions as two sensors and an ultraviolet irradiation light source. The freshness maintenance system 301 is configured with one semiconductor chip integrating three chips, each having a different function.

FIG. 6 shows the configuration of a freshness maintenance system 401 according to a fifth example. The housing 100 shown in FIG. 6 accommodates a circuit board 10 which is connected to a semiconductor chip 151 that constitutes the freshness maintenance unit 105 that functions as a freshness detection unit and a maturation suppression unit. The semiconductor chip 151 is a sensor element that functions as an ethylene sensor, and an ultraviolet light irradiation source. In other words, the ethylene sensor and the ultraviolet light irradiation unit are mounted on a single semiconductor chip. The freshness maintenance unit 105 includes a built-in semiconductor chip 151 having two functions and is packaged in the casing 100. The freshness maintenance system 401 is configured to include one casing that accommodates a one-chip semiconductor that functions as one sensor and an ultraviolet irradiation light source. The freshness maintenance system 401 is configured with one semiconductor chip integrating two chips, each having a different function.

FIG. 7 shows the configuration of a freshness maintenance system 501 according to a sixth example. The housing 100 shown in FIG. 7 accommodates a circuit board 10 which is connected to a semiconductor chip 251 that constitutes the freshness maintenance unit 205 that functions as a freshness detection unit and a maturation suppression unit. The semiconductor chip 251 is a sensor element that functions as a smell sensor, and an ultraviolet light irradiation source. In other words, the smell sensor and the ultraviolet light irradiation unit are mounted on a single semiconductor chip. The freshness maintenance unit 205 includes a built-in semiconductor chip 251 having two functions and is packaged in the casing 100. The freshness maintenance system 501 is configured to include one casing that accommodates a one-chip semiconductor that functions as one sensor and an ultraviolet irradiation light source. The freshness maintenance system 501 is configured with one semiconductor chip integrating two chips, each having a different function.

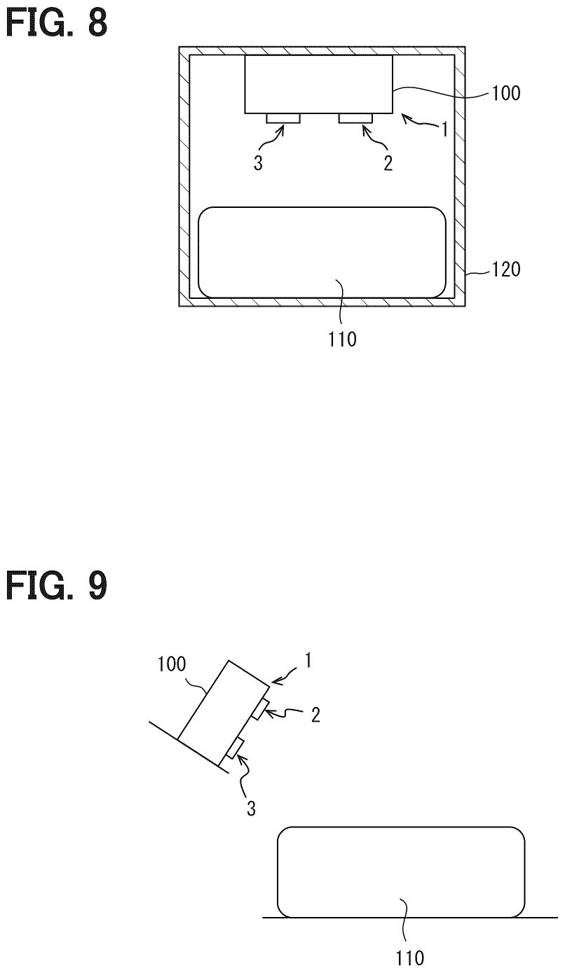

FIG. 8 shows a state in which the freshness maintenance system 1 performs freshness control on an agricultural product 110 accommodated in a cardboard box 120 or a container. The freshness maintenance system 1 is disposed in a state where the detection portion of the freshness detection unit 2 and the irradiation portion of the maturation suppression unit 3 face the agricultural product 110.

FIG. 9 shows a state in which the freshness maintenance system 1 performs the freshness control on the agricultural product placed in an open space. The freshness maintenance system 1 is placed at a height diagonally above the agricultural product 110. The detection portion of the freshness detection unit 2 and the irradiation portion of the maturation suppression unit 3 face the agricultural product 110. The freshness maintenance system 1 is placed in a state where the freshness of the agricultural product 110 stored on a stand such as a pallet or on display in a store can be controlled.

The freshness maintenance system 1 may be configured to use the sterilizing effect of ethanol instead of the ultraviolet light irradiation unit. The ethanol may be preferably applied to the wall surfaces or the like located around the agricultural product 110.

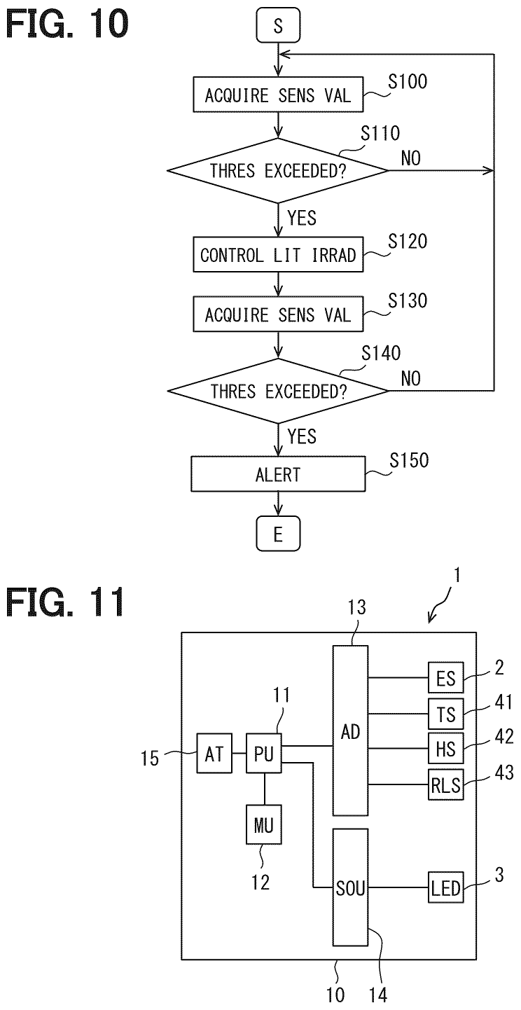

The freshness maintenance system executes freshness maintenance control in accordance with the flowchart shown in FIG. 10. The CPU mounted on the circuit board 10 executes the process shown in FIG. 10 and functions as a processing unit 11 and other units.

The process shown in FIG. 10 is executed when the electric power is supplied to the control circuit or when a predetermined time has elapsed after the electric power is supplied, and is repeated, for example, several times a day. In step S100, the acquisition unit 13 acquires a detection value of the sensor functioning as the freshness detection unit 2. In step S110, the processing unit 11 determines whether or not the detection value of the sensor is greater than a threshold value. The threshold value is preliminarily set to the upper limit value at which the maturation of the agricultural product can be maintained at a good quality or a value slightly lower than the upper limit value. In other words, the agricultural product for which the detection value of the sensor exceeds the threshold value is in an excellent condition and just before reaching a spoil level. The threshold value is set according to, for example, the time until the agricultural product subject to freshness maintenance control is ready to be sold as a sales product or until the agricultural product is eaten by a consumer. Thus, it is possible to execute the maturation suppression control with respect to the agricultural product that is still of good quality even if the detection value of the sensor exceeds the threshold value, and to take measures before the agricultural product reaches the spoil level.

If the sensor is an ethylene sensor, in step S110, it is determined whether the detected ethylene concentration exceeds a threshold value. If the sensor is an odor sensor, in step S110, it is determined whether or not the detected value of a parameter related to a specific smell component exceeds a threshold value.

The determination process of step S110 is repeatedly performed on newly acquired sensor detection values until the sensor detection value exceeds the threshold value. If it is determined in step S110 that the detected value of the sensor exceeds the threshold value, light irradiation control is executed in step S120. The processing unit 11 determines the amount of irradiation, the intensity of irradiation, and the range of irradiation according to the result of comparing the detection value of the sensor with a threshold value. The processing unit 11 determines the amount of irradiation, the irradiation intensity, and the irradiation range to be larger as the difference between the detection value of the sensor and the threshold value becomes larger. The irradiation unit 14 outputs a control signal to the maturation suppression unit 3 for executing the irradiation with the irradiation amount and the like determined by the processing unit 11. Furthermore, the processing unit 11 may be configured to determine the wavelength of the irradiation light according to the result of comparison between the detection value of the sensor and a threshold value.

The process of step S120 may be a process of controlling the amount of irradiation or the intensity of irradiation depending on the detection value of the temperature sensor 41. If the detection value of the temperature sensor 41 is high, maturation or spoilage will accelerate, so that, in the process of step S120, the amount of irradiation or the intensity of irradiation is controlled to be large. For example, when the detection value of the temperature sensor 41 exceeds the standard value, the amount of irradiation or the intensity of irradiation is controlled to be larger than when the detection value is below the standard value. For example, the amount of irradiation and the intensity of irradiation are controlled to be large as the detection value of the temperature sensor 41 increases. If the detection value of the temperature sensor 41 is low, the maturation or spoilage process will slow down, so that, in the process of step S120, the amount of irradiation or the intensity of irradiation is controlled to be small. For example, when the detection value of the temperature sensor 41 is below the standard value, the amount of irradiation or the intensity of irradiation is controlled to be smaller than when the detection value is above the reference value. For example, the amount of irradiation and the intensity of irradiation are controlled to be smaller as the detection value of the temperature sensor 41 becomes lower.

The process of step S120 may be a process of controlling the amount of irradiation or the intensity of irradiation according to the detection value of the humidity sensor 42. If the detection value of the humidity sensor 42 is high, the maturation or spoilage will accelerate, so that, in the process of step S120, the amount of irradiation or the intensity of irradiation is controlled to be large. For example, when the detection value of the humidity sensor 42 exceeds the standard value, the amount of irradiation or the intensity of irradiation is controlled to be larger than when the detection value is below the standard value. For example, the amount of irradiation and the intensity of irradiation are controlled to be large as the detection value of the humidity sensor 42 increases. If the detection value of the humidity sensor 42 is low, the maturation or spoilage process will slow down, so that, in the process of step S120, the amount of irradiation or the intensity of irradiation is controlled to be small. For example, when the detection value of the humidity sensor 42 is below the standard value, the amount of irradiation or the intensity of irradiation is controlled to be smaller than when the detection value is above the reference value. For example, the amount of irradiation and the intensity of irradiation are controlled to be smaller as the detection value of the humidity sensor 42 becomes lower.

By performing the process of step S120, the decrease in the freshness of the agricultural product can be suppressed at the timing when the freshness of the agricultural product starts to decrease, thereby it is possible to maintain the freshness. For example, the period from when the agricultural product is shipped until the freshness of the agricultural product starts to decrease can be extended, thereby enabling the period during which the freshness can be maintained to be extended.

In step S130, the acquisition unit 13 acquires the sensor detection value for the agricultural product that has been subjected to the maturation suppression effect in step S120. In step S140, the processing unit 11 determines whether or not the detection value of the sensor is greater than a threshold value. The threshold value in step S140 may be set to a value equivalent to the threshold value in step S110. The threshold value in step S140 may be set to a value lower than the threshold value in step S110.

The determination process of step S140 is repeatedly performed on newly acquired sensor detection values with performing the light irradiation control until the sensor detection value exceeds the threshold value. If the sensor detection value exceeds the threshold value in step S140, the sensor detection value will not decrease to a level at which the freshness can be maintained. In this case, the freshness maintenance system 1 generates an alert in step S150 and ends the flow chart of FIG. 10. The alert in step S150 notifies the user or an administrator that the agricultural produce proceeds the spoilage, so that the user or administrator can take necessary measures. For example, the user and the like can reduce waste loss by changing from fresh foods to processed foods or by using the data as a price reduction indicator.

In step S150, the output unit 15 wirelessly communicates with an external device or the cloud system. An external device such as a mobile terminal or a personal computer is configured to notify the alert information acquired from the output unit 15 by displaying on a display screen or by outputting a sound or the like. Furthermore, the freshness maintenance system 1 may be configured to notify the alert information by displaying on a display screen or by outputting the sound or the like in a device constituting the system.

The determination process in step S110 may be configured to execute the process in step S120 according to an estimate of the degree of maturation or the degree of spoilage of the agricultural product based on the detection value of the temperature sensor 41. This estimation control using the temperature environment makes it possible to provide feedforward control rather than the maturation suppression processing based on actually measured maturation of the agricultural product.

The determination process in step S110 may be configured to execute the process in step S120 according to an estimate of the degree of maturation or the degree of spoilage of the agricultural product based on the detection value of the humidity sensor 42. This estimation control using the humidity environment makes it possible to provide feedforward control rather than the maturation suppression processing based on actually measured maturation of the agricultural product.

The freshness maintenance system 1 of the first embodiment includes a freshness detection unit 2 that detects the freshness of the agricultural produce by detecting a specific gas, and a maturation suppression unit 3 that irradiates light having a sterilizing effect. The freshness maturation system 1 includes a control unit that controls the maturation suppression unit 3 based on the freshness detected by the freshness detection unit 2 to suppress the progress of maturation of the agricultural product.

According to this system, the freshness detection unit 2 can detect the maturation and the spoiled smell of the agricultural product, and light irradiation that suppresses the progression of maturation can be executed according to the detected freshness state. This makes it possible to restrict the agricultural product from the maturation during storage or display in a store.

The control unit issues an alert if the maturation of the agricultural product progresses although the process of controlling the maturation suppression unit 3 is executed to suppress the maturation progress of the agricultural product. According to this control, if the degree of maturation does not decrease, it is possible to notify the user or the like that appropriate measures need to be taken for the agricultural product. This will enable the management to be implemented to suppress the food waste loss, and the like.

### Second Embodiment

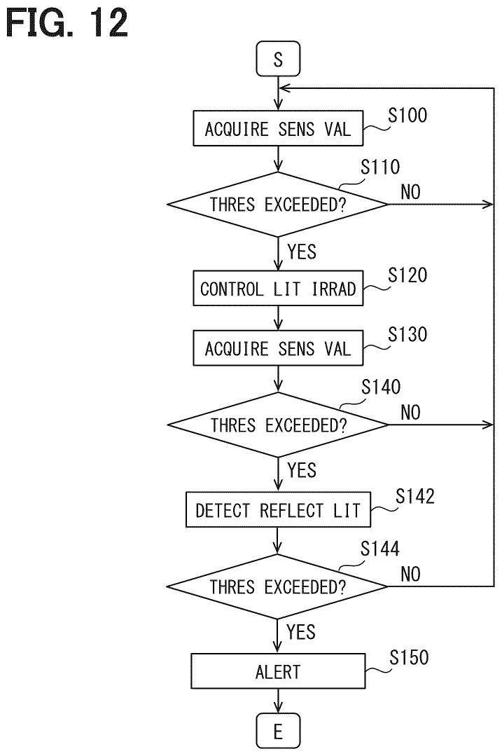

A second embodiment will be described with reference to FIGS. 11 to 12. The freshness maintenance system 1 of the second embodiment differs from the first embodiment in that the freshness maintenance system 1 includes a reflection light detection unit 43 that detects reflection light of the irradiation light, and in the accompanying freshness maintenance control. Configurations, operations, and effects that are not particularly described in the second embodiment are the same as those of the above-described embodiment, and different points will be described below.

As shown in FIG. 11, the freshness maintenance system 1 includes a reflection light detection unit 43 that detects reflection light of light irradiated by the maturation suppression unit 3. In the drawings, the reflection light detection unit 43 is indicated as RLS. The reflection light detection unit 43 includes a semiconductor element for receiving light that detects the intensity of the reflection light. The semiconductor element is mounted on a circuit board 10. The detection value detected by the reflection light detection unit 43 is input to the acquisition unit 13. The processing unit 11 determines the intensity of the reflection light based on the detection value of the reflection light acquired by the acquisition unit 13.

The freshness maintenance system according to the second embodiment executes freshness maintenance control in accordance with the flowchart shown in FIG. 12. The CPU mounted on the circuit board 10 executes the process shown in FIG. 12 and functions as a processing unit 11 and other units.

The process shown in FIG. 12 is executed when the electric power is supplied to the control circuit or when a predetermined time has elapsed after the electric power is supplied, and is repeated, for example, several times a day. The following describes the differences from the control described above with reference to FIG. 10 in the first embodiment. In each processing step shown in FIG. 12, steps having the same reference numerals as those shown in FIG. 10 are the same as those in the first embodiment, and therefore the description thereof will be omitted.

If the detection value of the sensor exceeds the threshold in step S140, the acquisition unit 13 acquires the amount of reflection light detected by the reflection light detection unit 43 in step S142. The processing unit 11 determines the intensity of the reflection light from the amount of the reflection light. In step S144, the processing unit 11 determines whether or not the intensity of the reflection light exceeds a threshold value. In step S140, since it is determined that the sensor detection value does not decrease sufficiently even after the ultraviolet light irradiation, in a further step S144, the amount of the ultraviolet light absorbed by the agricultural product is determined. If the intensity of the reflection light is high, it is determined that the agricultural product is unable to absorb sufficiently the ultraviolet light, and the effect of the ultraviolet light irradiation in step S120 is insufficient. If the intensity of the reflection light is low, it is determined that the ultraviolet light irradiation in step S120 is effective. In the determination process in step S144, it is possible to detect the freshness even if the sensor value detection in step S130 does not function normally or the sensor itself is failed.

The determination process in step S144 is repeatedly executed for the intensity of the reflection light newly acquired through the light irradiation control until the intensity of the reflection light exceeds the threshold value. If the intensity of the reflection light exceeds the threshold in step S144, the effect of maintaining the freshness cannot be obtained. In this case, the freshness maintenance system 1 generates an alert in step S150 and ends the flow chart of FIG. 12.

The freshness maintenance system of the second embodiment further includes a reflection light detection unit 43 that detects the reflection light of the light irradiated by the maturation suppression unit 3. The control unit determines whether to continue the light irradiation by the maturation suppression unit 3 or to generate an alert, depending on the level of the reflection light detected by the reflection light detection unit 43. According to this control, when the sensor value does not improve, it is possible to check whether the irradiation light is absorbed or reflected by the agricultural product, and it is possible to provide an index for determining the freshness of the agricultural product.

### Third Embodiment

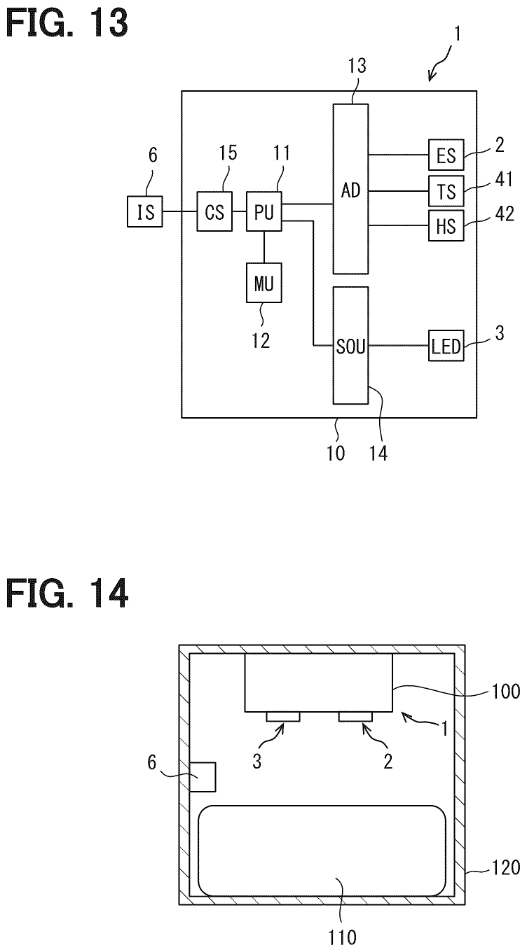

A third embodiment will be described with reference to FIGS. 13 to 15. The freshness maintenance system 1 of the third embodiment differs from the first embodiment in that freshness maintenance control is performed using images of the agricultural product. Configurations, operations, and effects that are not particularly described in the third embodiment are the same as those of the above-described embodiment, and different points will be described below.

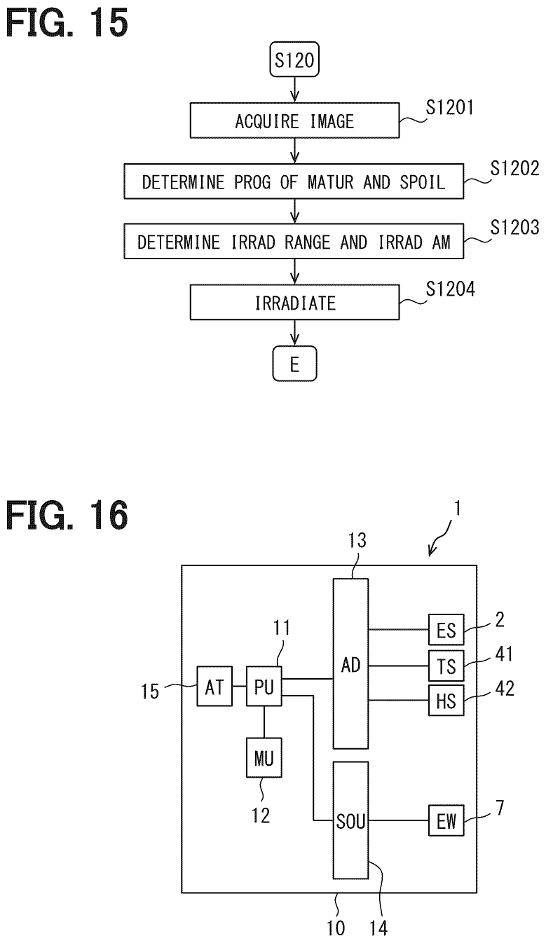

As shown in FIG. 13, the freshness maintenance system 1 acquires an image of the agricultural product captured by a camera 6, and uses the image to execute the control shown in the flowchart of FIG. 15. In the drawings, the camera 6 is indicated as IS. The freshness maintenance system 1 may be configured to include the camera 6 or may not include the camera 6. The acquisition unit 13 acquires the image of the agricultural product captured by the camera 6. The camera 6 is accommodated in a cardboard box, a container, or the like, as shown in FIG. 14. The camera 6 may be configured to be disposed in a state in which the camera 6 can capture the image of the agricultural product placed in an open space.

The freshness maintenance system 1 of the third embodiment executes a subroutine shown in FIG. 15 in the light irradiation control of step S120. The CPU mounted on the circuit board 10 executes the process shown in FIGS. 10 and 15 and functions as a processing unit 11 and other units.

The following describes the differences from the control described above with reference to FIG. 10 in the first embodiment. The freshness maintenance control according to the third embodiment is similar to the steps shown in FIG. 10 except for the processing steps shown in FIG. 15 in step S120, and therefore description thereof will be omitted.

In step S1201, the acquisition unit 13 acquires image data of the agricultural product captured by the camera 6. In step S1202, the processing unit 11 analyzes the image captured by the camera 6 to estimate and determine the degree of maturation or the progress of spoilage of the agricultural product. The processing unit 11 estimates the degree of maturation or the progress of the spoilage by using AI image recognition and determination that uses machine learning to learn the color, gloss, lighting conditions, and the like of the agricultural product based on the analyzed image. This estimation process enables the processing unit 11 to estimate particularly the degree of the maturation or the progress of the spoilage of the agricultural product. In the AI image recognition determination, the level of the state of the agricultural product is determined for each part of the agricultural product on a multi-level scale. Furthermore, characteristic data indicating the relationship between the image data and the degree of maturation or the progress of spoilage of the agricultural product is stored, for example, in the memory unit 12.

In step S1203, the processing unit 11 determines the light irradiation range, the irradiation time, the amount of irradiation, and the intensity of irradiation on the agricultural product based on the determined degree of maturation or determined progress of spoilage of the agricultural product. In step S1204, the irradiation unit 14 outputs a control signal to the maturation suppression unit 3 for controlling the light irradiation range, the irradiation time, the irradiation amount, the intensity, and the like determined by the processing unit 11. The freshness maintenance system 1 ends the subroutine of step S120 and proceeds to step S130.

Alternatively, it may be possible to determine whether or not to execute the alert process in step S150 based on the degree of maturation of the agricultural product estimated using the image of the agricultural product taken by the camera 6. According to this control, since the determination is made not only based on the sensor detection value but also based on image recognition, the accuracy of the alert processing is improved, and the user can accurately take appropriate measures.

The freshness maturation system 1 of the third embodiment includes a camera 6 that takes the image of the agricultural product. The control unit controls the irradiation range and the irradiation intensity of the light based on the degree of maturation of the agricultural product estimated using the image of the agricultural product taken by the camera 6. According to this control, the freshness of the agricultural product can be detected for each part or particularly based on the image of the agricultural product. According to this detection, the irradiation range of the ultraviolet light can be limited, so that the irradiation time can be shortened and the irradiation energy can be saved. Furthermore, according to the control of the third embodiment, the freshness state of the agricultural product can be detected for each part or particularly, thereby improving the accuracy of the alert process in step S150.

### Fourth Embodiment

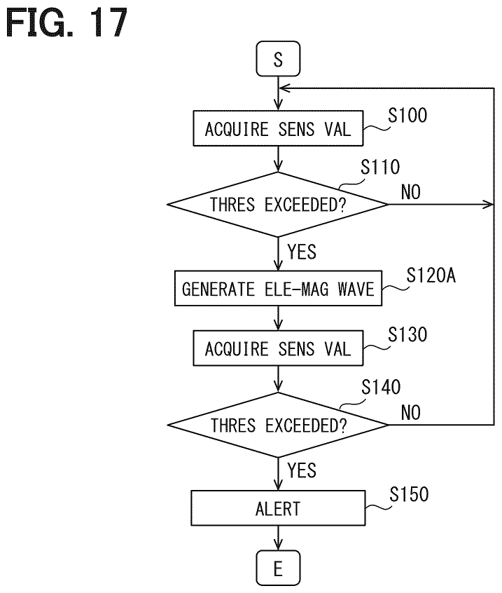

A fourth embodiment will be described with reference to FIGS. 16 to 17. The freshness maintenance system 1 of the fourth embodiment differs from the first embodiment in that the maturation is suppressed by using an electromagnetic wave. Configurations, operations, and effects that are not particularly described in the fourth embodiment are the same as those of the above-described embodiment, and different points will be described below.

As shown in FIG. 16, freshness maintenance system 1 includes an electromagnetic wave generation unit 7 which is a maturation suppression unit that suppresses the progress of maturation by the electromagnetic wave. In the drawings, the electromagnetic wave generation unit 7 is indicated as EW. An example of the electromagnetic wave generation unit 7 will be described below. The electromagnetic wave generation unit 7 includes a photoconductive substrate on which a thin film of photoconductive material is stacked, and an antenna formed on the photoconductive substrate. The antenna is a dipole antenna including a pair of electrode portions and a pair of antenna bodies. When a voltage is applied to the pair of antenna bodies and excitation light such as a femtosecond pulse laser is irradiated onto the gap between the pair of antenna bodies, photo-excited carriers are generated. When a pulsed current flows through the gap between the pair of antenna bodies, the electromagnetic wave generation unit 7 generates a terahertz electromagnetic wave by this current.

The process shown in FIG. 17 is executed when the electric power is supplied to the control circuit or when a predetermined time has elapsed after the electric power is supplied, and is repeated, for example, several times a day. The following describes the differences from the control described above with reference to FIG. 10 in the first embodiment. In each processing step shown in FIG. 17, steps having the same reference numerals as those shown in FIG. 10 are the same as those in the first embodiment, and therefore the description thereof will be omitted.

If it is determined in step S110 that the detection value of the sensor exceeds the threshold value, electromagnetic wave generation control is executed in step S120A. The processing unit 11 determines the amount and the strength of the electromagnetic wave to be generated according to the result of comparing the detection value of the sensor with a threshold value. The processing unit 11 determines that the amount and the intensity of electromagnetic wave to be generated are increased as the difference between the detection value of the sensor and the threshold value increases. The irradiation unit 14 outputs a control signal to the electromagnetic wave generation unit 7 for controlling the amount and the intensity of the electromagnetic wave determined by the processing unit 11. The generated electromagnetic wave promote the electronic transition of ethylene molecules around the agricultural product and change their electronic bonds, thereby contributing to the removal of ethylene molecules. In this way, the electromagnetic wave generation unit 7 removes ethylene molecules and provides a sterilizing effect. According to step S120, a maturation suppression effect can be provided to the agricultural product, similar to the ultraviolet light irradiation in the first embodiment.

The process of step S120A may be a process of controlling the intensity of the electromagnetic wave in accordance with the detection value of the temperature sensor 41. If the detection value of the temperature sensor 41 is high, maturation or spoilage will accelerate, so that, in the process of step S120A, the intensity of the electromagnetic wave is controlled to be large. For example, when the detection value of the temperature sensor 41 exceeds the standard value, the intensity of the electromagnetic wave is controlled to be larger than when the detection value is below the standard value. For example, the intensity of the electromagnetic waves is controlled to be greater as the detection value of the temperature sensor 41 increases. If the detection value of the temperature sensor 41 is low, the maturation or spoilage process will slow down, so that, in the process of step S120A, the intensity of the electromagnetic wave is controlled to be small. For example, when the detection value of the temperature sensor 41 is below the standard value, the intensity of the electromagnetic wave is controlled to be smaller than when the detection value is above the reference value. For example, the intensity of the electromagnetic waves is controlled to be smaller as the detection value of the temperature sensor 41 becomes lower.

The process of step S120A may be a process of controlling the intensity of the electromagnetic wave in accordance with the detection value of the humidity sensor 42. If the detection value of the humidity sensor 42 is high, maturation or spoilage will accelerate, so that, in the process of step S120A, the intensity of the electromagnetic wave is controlled to be large. For example, when the detection value of the humidity sensor 42 exceeds the standard value, the intensity of the electromagnetic wave is controlled to be larger than when the detection value is below the standard value. For example, the intensity of the electromagnetic waves is controlled to be greater as the detection value of the humidity sensor 42 increases. If the detection value of the humidity sensor 42 is low, the maturation or spoilage process will slow down, so that, in the process of step S120A, the intensity of the electromagnetic wave is controlled to be small. For example, when the detection value of the humidity sensor 42 is below the standard value, the intensity of the electromagnetic wave is controlled to be smaller than when the detection value is above the reference value. For example, the intensity of the electromagnetic waves is controlled to be smaller as the detection value of the humidity sensor 42 becomes lower.

The freshness maintenance system 1 of the fourth embodiment includes a freshness detection unit 2 that detects the freshness of the agricultural product by detecting a specific gas, and a maturation suppression unit that generates the electromagnetic wave. The freshness maturation system includes a control unit that controls the maturation suppression unit based on the freshness detected by the freshness detection unit 2 to suppress the progress of maturation of the agricultural product. According to this system, the freshness detection unit 2 can detect the maturation and the spoiled smell of the agricultural product, and the electromagnetic wave that suppresses the progression of maturation can be generated according to the detected freshness state. By removing ethylene molecules using the electromagnetic wave, it is possible to restrict the maturation of the agricultural product during storage or display in a store.

### Fifth Embodiment

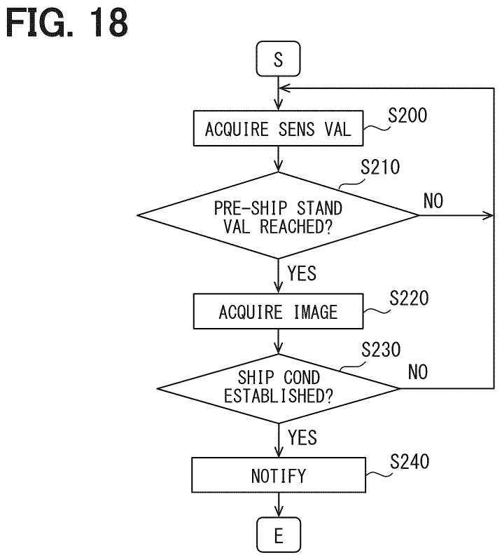

A fifth embodiment will be described with reference to FIG. 18. The freshness maintenance system of the fifth embodiment differs from the above-described embodiments in that the freshness maintenance system further performs control to notify the user that the shipping conditions for the agricultural product have been established. Configurations, operations, and effects that are not particularly described in the fifth embodiment are the same as those of the above-described embodiment, and different points will be described below.

The freshness maintenance system 1 executes freshness maintenance control according to the flowchart shown in FIG. 18 in addition to the control according to the flowchart described above. The freshness maintenance system of the fifth embodiment executes the control according to the flowchart of FIG. 18 in parallel with the respective flowcharts of FIG. 10, FIG. 12, FIG. 15 and FIG. 17. The CPU mounted on the circuit board 10 executes the process shown in FIG. 18 and functions as a processing unit 11 and other units.

The process shown in FIG. 18 is executed when the electric power is supplied to the control circuit or when a predetermined time has elapsed after the electric power is supplied, and is repeated, for example, several times a day. By executing the control shown in FIG. 18, the maturation of the agricultural product can be monitored using a smell sensor and an ethylene sensor while the state of after-ripening can be monitored based on the camera image, and it is possible to detect a state immediately before full ripeness.

In step S200, the acquisition unit 13 acquires a detection value of the smell sensor functioning as the freshness detection unit 2. In step S210, the processing unit 11 determines whether or not the detection value of the smell sensor has reached the pre-shipment standard value. The pre-shipment standard value is a value lower than the threshold value in step S110 of the first embodiment, and is preliminarily set to a value at which the agricultural product is in a state immediately before the full ripeness. If it is determined that the detection value of the smell sensor has reached the pre-shipment standard value, then in step S220, the acquisition unit 13 acquires image data of the agricultural product captured by the camera 6. In step S230, the processing unit 11 determines whether the shipment conditions have been established by AI image recognition determination, which uses machine learning to learn the color, gloss, lighting conditions, and the like of the agricultural product based on the analyzed image. Through this determination process, the processing unit 11 can detect the shipment status of the agricultural product with high accuracy by visually determining the maturation using the image after grasping the degree of after-ripening using the smell sensor. Furthermore, characteristic data indicating the relationship between the image data and the establishment of the shipment conditions is stored in, for example, the memory unit 12.

The determination process in step S230 is repeatedly executed via the determination process in step S210 and image acquisition until the shipment conditions are established. If the shipment conditions are established as a result of the determination process in step S230, the freshness maintenance system 1 executes a notification process in step S240, and then ends the flow chart of FIG. 18. The notification in step S240 indicating that the agricultural product is in a proper shipment state is recognized to the user or the administrator, so that the user or administrator can take steps to prepare for shipping.

In step S230, the output unit 15 wirelessly communicates with an external device or the cloud system. An external device such as a mobile terminal or a personal computer is configured to notify the shipment ready state acquired from the output unit 15 by displaying on a display screen or by outputting a sound or the like. Furthermore, the freshness maintenance system 1 may be configured to notify the shipment ready state by displaying on a display screen or by outputting the sound or the like in a device constituting the system.

The freshness maintenance system of the fifth embodiment may be configured so that ethylene gas is sprayed onto walls or the like located around the agricultural product. The freshness maintenance system may also be configured to supply ethylene gas into a box accommodating the agricultural product. This configuration can promote the maturation of the agricultural product, contributing to increased after-ripening of the agricultural product.

The control unit of the fifth embodiment notifies that the conditions for shipping the agricultural product have been established based on the degree of maturation of the agricultural product detected by the freshness detection unit 2 and the degree of maturation of the agricultural product estimated using image of the agricultural product taken by the camera 6. According to this, the proper shipment time can be determined with high accuracy by determining the shipment conditions based on a combination of the degree of maturation detected by the smell sensor or the ethylene sensor and the degree of maturation determined by image recognition.

### Other Embodiments

The disclosure of the present description is not restricted to the embodiments shown as examples. The present disclosure includes embodiments described above and modifications of the above-described embodiments made by a person skilled in the art. For example, the disclosure is not limited to a combination of components and elements described in the embodiments, and various modifications may be implemented. The present disclosure may be implemented in various combinations thereof. The disclosure may have additional components that can be added to the embodiments. The disclosure encompasses the omission of parts and elements of the embodiments. The disclosure encompasses the replacement or combination of components, elements between one embodiment and another. The disclosed technical scope is not limited to the description of the embodiments. The disclosed technical scope is indicated by the description of the claims, and should be understood to include meanings equivalent to the description of the claims and all modifications within the scope.

The control device and the method therefor which have been described in the present disclosure may be also realized by a dedicated computer which constitutes a processor programmed to execute one or more functions concretized by computer programs. Alternatively, the device and the method described in the present disclosure may be implemented by a dedicated hardware logic circuit. Alternatively, the device and the method thereof described in the present disclosure may be implemented by one or more dedicated computers configured by a combination of a processor that executes a computer program and one or more hardware logic circuits. The computer program may also be stored on a computer-readable and non-transitory tangible storage medium as an instruction executed by a computer.

The freshness maintenance system 1 of the first embodiment may be configured to include either a temperature sensor or a humidity sensor. Furthermore, the freshness maintenance system 1 of the first embodiment may be configured without including a temperature sensor and a humidity sensor.

### (Technical Features)

This specification discloses multiple technical features described in multiple items listed below. Some features may be described in a multiple dependent form, in which subsequent features alternatively refer to preceding features. These features described in a multiple dependent form define multiple technical features.

### (Technical Feature 1)

A freshness maintenance system includes: at least one of (i) a circuit and (ii) a processor having a memory storing computer program code. The at least one of the circuit and the processor having the memory is configured to cause the freshness maintenance system to provide: a freshness detection unit that detects a specific gas to detect a freshness of an agricultural product; a maturation suppression unit that irradiates light having a sterilizing effect; and a control unit that controls the maturation suppression unit based on the freshness detected by the freshness detection unit to suppress a progress of maturation of the agricultural product.

### (Technical Feature 2)

A freshness maintenance system includes: a freshness detection unit that detects a specific gas to detect a freshness of an agricultural product; a maturation suppression unit that generates an electromagnetic wave; and a control unit that controls the maturation suppression unit based on the freshness detected by the freshness detection unit to suppress a progress of maturation of the agricultural product.

### (Technical Feature 3)

In the freshness maintenance system according to technical feature 1 or 2, the control unit controls an intensity of light irradiation or an intensity of the electromagnetic wave so as to suppress the maturation of the agricultural product in response to temperature detected by a temperature sensor.

### (Technical Feature 4)

In the freshness maintenance system according to technical feature 1 or 2, the control unit controls an intensity of light irradiation or an intensity of the electromagnetic wave so as to suppress the maturation of the agricultural product in response to humidity detected by a humidity sensor.

### (Technical Feature 5)

In the freshness maintenance system according to any one of technical features 1 to 4, the control unit generates an alert if the maturation of the agricultural product progresses although a process of controlling the maturation suppression unit 3 is executed to suppress the maturation of the agricultural product.

### (Technical Feature 6)

The freshness maintenance system according to any one of technical features 1 to 4, further includes: a reflection light detection unit that detects reflection light of the light irradiated by the maturation suppression unit. The control unit determines whether to continue irradiating the light by the maturation suppression unit or to generate an alert, depending on a level of the reflection light detected by the reflection light detection unit.

### (Technical Feature 7)

In the freshness maintenance system according to any one of technical features 1 to 4, the control unit controls an irradiation range and an irradiation intensity of the light based on a degree of the maturation of the agricultural product estimated using an image of the agricultural product taken by a camera.

### (Technical Feature 8)

In the freshness maintenance system according to any one of technical features 1 to 7, the control unit notifies that a shipment condition of the agricultural product have been established based on a degree of the maturation of the agricultural product detected by the freshness detection unit and a degree of the maturation of the agricultural product estimated using an image of the agricultural product taken by a camera.

### (Technical Feature 9)

In the freshness maintenance system according to any one of technical features 1 to 8, the freshness detection unit includes a semiconductor chip equipped with an ethylene sensor for detecting a concentration of ethylene and a smell sensor for detecting a specific smell component.

### (Technical Feature 10)

In the freshness maintenance system according to any one of technical features 1 to 8, the freshness detection unit includes an ethylene sensor for detecting a concentration of ethylene and a smell sensor for detecting a specific smell component.

The maturation suppression unit includes an ultraviolet light irradiation unit that irradiates UV-A or UV-B; and the ethylene sensor, the smell sensor, and the ultraviolet light irradiation unit are mounted on a single semiconductor chip.

### (Technical Feature 11)

In the freshness maintenance system according to any one of technical features 1 to 8, the freshness detection unit includes an ethylene sensor for detecting a concentration of ethylene; the maturation suppression unit includes an ultraviolet light irradiation unit that irradiates UV-A or UV-B; and the ethylene sensor and the ultraviolet light irradiation unit are mounted on a single semiconductor chip.

### (Technical Feature 12)

In the freshness maintenance system according to any one of technical features 1 to 8, the freshness detection unit includes a smell sensor for detecting a specific smell component; the maturation suppression unit includes an ultraviolet light irradiation unit that irradiates UV-A or UV-B; and the smell sensor and the ultraviolet light irradiation unit are mounted on a single semiconductor chip.

### (Technical Feature 13)

In the freshness maintenance system according to any one of technical features 1 to 8, the freshness detection unit includes an ethylene sensor for detecting a concentration of ethylene.

### (Technical Feature 14)

In the freshness maintenance system according to any one of technical features 1 to 8, the freshness detection unit includes a smell sensor for detecting a specific smell component.

### (Technical Feature 15)

In the freshness maintenance system according to any one of technical features 1 to 8, the maturation suppression unit includes an ultraviolet light irradiation unit that irradiates UV-A or UV-B.

It is noted that a flowchart or the processing of the flowchart in the present application includes sections (also referred to as steps), each of which is represented, for instance, as S100. Further, each section can be divided into several sub-sections while several sections can be combined into a single section. Furthermore, each of thus configured sections can be also referred to as a device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A freshness maintenance system comprising:
a freshness detection unit that detects a specific gas to detect a freshness of an agricultural product;
a maturation suppression unit that irradiates light having a sterilizing effect or generates an electromagnetic wave; and
a control unit that controls the maturation suppression unit based on the freshness detected by the freshness detection unit to suppress a progress of maturation of the agricultural product, wherein:
the control unit controls an intensity of light irradiation or an intensity of the electromagnetic wave to be larger in a case where temperature detected by a temperature sensor exceeds a predetermined value than in a case where the temperature does not exceed the predetermined value.

2. The freshness maintenance system according to claim 1, wherein:
the freshness detection unit includes a semiconductor chip equipped with an ethylene sensor for detecting a concentration of ethylene and a smell sensor for detecting a specific smell component.

3. The freshness maintenance system according to claim 1, wherein:
the freshness detection unit includes an ethylene sensor for detecting a concentration of ethylene and a smell sensor for detecting a specific smell component;
the maturation suppression unit includes an ultraviolet light irradiation unit that irradiates UV-A or UV-B; and
the ethylene sensor, the smell sensor, and the ultraviolet light irradiation unit are mounted on a single semiconductor chip.

4. The freshness maintenance system according to claim 1, wherein:
the freshness detection unit includes an ethylene sensor for detecting a concentration of ethylene;
the maturation suppression unit includes an ultraviolet light irradiation unit that irradiates UV-A or UV-B; and
the ethylene sensor and the ultraviolet light irradiation unit are mounted on a single semiconductor chip.

5. The freshness maintenance system according to claim 1, wherein:
the freshness detection unit includes a smell sensor for detecting a specific smell component;
the maturation suppression unit includes an ultraviolet light irradiation unit that irradiates UV-A or UV-B; and
the smell sensor and the ultraviolet light irradiation unit are mounted on a single semiconductor chip.

6. The freshness maintenance system according to claim 1, wherein:
the freshness detection unit includes an ethylene sensor for detecting a concentration of ethylene.

7. The freshness maintenance system according to claim 1, wherein:
the freshness detection unit includes a smell sensor for detecting a specific smell component.

8. The freshness maintenance system according to claim 1, wherein:
the maturation suppression unit includes an ultraviolet light irradiation unit that irradiates UV-A or UV-B.

9. The freshness maintenance system according to claim 1, further comprising:
at least one of (i) a circuit and (ii) a processor having a memory storing computer program code, wherein:
the at least one of the circuit and the processor having the memory is configured to cause the freshness maintenance system to provide at least one of: the freshness detection unit; the maturation suppression unit; and the control unit.

10. A freshness maintenance system comprising:
a freshness detection unit that detects a specific gas to detect a freshness of an agricultural product;
a maturation suppression unit that irradiates light having a sterilizing effect or generates an electromagnetic wave; and
a control unit that controls the maturation suppression unit based on the freshness detected by the freshness detection unit to suppress a progress of maturation of the agricultural product, wherein:
the control unit controls an intensity of light irradiation or an intensity of the electromagnetic wave to be larger in a case where humidity detected by a humidity sensor exceeds a predetermined value than in a case where the humidity does not exceed the predetermined value.

11. The freshness maintenance system according to claim 10, wherein:
the freshness detection unit includes a semiconductor chip equipped with an ethylene sensor for detecting a concentration of ethylene and a smell sensor for detecting a specific smell component.

12. The freshness maintenance system according to claim 10, wherein:
the freshness detection unit includes an ethylene sensor for detecting a concentration of ethylene and a smell sensor for detecting a specific smell component;
the maturation suppression unit includes an ultraviolet light irradiation unit that irradiates UV-A or UV-B; and
the ethylene sensor, the smell sensor, and the ultraviolet light irradiation unit are mounted on a single semiconductor chip.

13. The freshness maintenance system according to claim 10, wherein:
the freshness detection unit includes an ethylene sensor for detecting a concentration of ethylene;
the maturation suppression unit includes an ultraviolet light irradiation unit that irradiates UV-A or UV-B; and
the ethylene sensor and the ultraviolet light irradiation unit are mounted on a single semiconductor chip.

14. The freshness maintenance system according to claim 10, wherein:
the freshness detection unit includes a smell sensor for detecting a specific smell component;
the maturation suppression unit includes an ultraviolet light irradiation unit that irradiates UV-A or UV-B; and
the smell sensor and the ultraviolet light irradiation unit are mounted on a single semiconductor chip.

15. The freshness maintenance system according to claim 10, wherein:
the freshness detection unit includes an ethylene sensor for detecting a concentration of ethylene.

16. The freshness maintenance system according to claim 10, wherein:
the freshness detection unit includes a smell sensor for detecting a specific smell component.

17. The freshness maintenance system according to claim 10, wherein:
the maturation suppression unit includes an ultraviolet light irradiation unit that irradiates UV-A or UV-B.

18. The freshness maintenance system according to claim 10, further comprising:
at least one of (i) a circuit and (ii) a processor having a memory storing computer program code, wherein:
the at least one of the circuit and the processor having the memory is configured to cause the freshness maintenance system to provide at least one of: the freshness detection unit; the maturation suppression unit; and the control unit.

19. A freshness maintenance system comprising:
a freshness detection unit that detects a specific gas to detect a freshness of an agricultural product;
a maturation suppression unit that irradiates light having a sterilizing effect;
a control unit that controls the maturation suppression unit based on the freshness detected by the freshness detection unit to suppress a progress of maturation of the agricultural product; and
a reflection light detection unit that detects reflection light of the light irradiated by the maturation suppression unit, wherein:
the control unit continues irradiating the light by the maturation suppression unit when a level of the reflection light detected by the reflection light detection unit falls below a threshold value; and
the control unit generate an alert when the level of the reflection light detected by the reflection light detection unit does not fall below the threshold value.

20. The freshness maintenance system according to claim 19, wherein:
the freshness detection unit includes a semiconductor chip equipped with an ethylene sensor for detecting a concentration of ethylene and a smell sensor for detecting a specific smell component.

21. The freshness maintenance system according to claim 19, wherein:
the freshness detection unit includes an ethylene sensor for detecting a concentration of ethylene and a smell sensor for detecting a specific smell component;
the maturation suppression unit includes an ultraviolet light irradiation unit that irradiates UV-A or UV-B; and
the ethylene sensor, the smell sensor, and the ultraviolet light irradiation unit are mounted on a single semiconductor chip.

22. The freshness maintenance system according to claim 19, wherein:
the freshness detection unit includes an ethylene sensor for detecting a concentration of ethylene;
the maturation suppression unit includes an ultraviolet light irradiation unit that irradiates UV-A or UV-B; and
the ethylene sensor and the ultraviolet light irradiation unit are mounted on a single semiconductor chip.

23. The freshness maintenance system according to claim 19, wherein:
the freshness detection unit includes a smell sensor for detecting a specific smell component;
the maturation suppression unit includes an ultraviolet light irradiation unit that irradiates UV-A or UV-B; and
the smell sensor and the ultraviolet light irradiation unit are mounted on a single semiconductor chip.

24. The freshness maintenance system according to claim 19, wherein:
the freshness detection unit includes an ethylene sensor for detecting a concentration of ethylene.

25. The freshness maintenance system according to claim 19, wherein:
the freshness detection unit includes a smell sensor for detecting a specific smell component.

26. The freshness maintenance system according to claim 19, wherein:
the maturation suppression unit includes an ultraviolet light irradiation unit that irradiates UV-A or UV-B.

27. The freshness maintenance system according to claim 19, further comprising:
at least one of (i) a circuit and (ii) a processor having a memory storing computer program code, wherein:
the at least one of the circuit and the processor having the memory is configured to cause the freshness maintenance system to provide at least one of: the freshness detection unit; the maturation suppression unit; and the control unit.
